(19) United States
(12) Patent Application Publication
(10) Pub. No.: US 2018/0372722 A1
(43) Pub. Date: Dec. 27, 2018

(54) METHOD FOR DETECTING miRNA-206 FOR ANALYZING THE DIAGNOSIS OR PROGNOSIS OF A MENTAL ILLNESS BY A MOOD DISORDER, METHOD FOR PROVIDING INFORMATION FOR THE DIAGNOSIS, AND A COMPOSITION FOR TARGETING miRNA-206

(71) Applicant: ADVANCED NT, Seoul (KR)

(72) Inventors: Kon CHU, Seoul (KR); Sang Kun LEE, Seoul (KR); Manho KIM, Seoul (KR); Keun-Hwa JUNG, Seoul (KR); Soon-Tae LEE, Seoul (KR); Daejong JEON, Daejeon (KR)

(21) Appl. No.: 16/063,558

(22) PCT Filed: Oct. 21, 2016
(86) PCT No.: PCT/KR2016/011934

§ 371 Date: Jun. 18, 2018

(30) Foreign Application Priority Data
Dec. 17, 2015 (KR) 10-2015-0180919

(51) Int. Cl. G01N 33/50; A61P 25/24; A61K 31/711; A61K 31/7105

(52) U.S. Cl. CPC G01N 33/5023; A61K 31/7105; A61K 31/711; A61P 25/24

(57) ABSTRACT

The present invention relates to a method for detecting miRNA-206 for analyzing the diagnosis or prognosis of a mental illness by a mood disorder, a method for providing information for the diagnosis, and a composition for targeting miRNA-206, more specifically, the present invention can detect diagnosis or prognostic analysis marker of the said mental illness by a mood disorder, enable a more accurate diagnosis of a mental illness by a mood disorder by measuring the expression level of miR-206 from a tissue sample of a patient, provide a diagnostic composition capable of measuring the expression level of the said miR-206, and furthermore provide a pharmaceutical composition capable of treating and preventing a mental illness by a mood disorder by using an oligonucleotide that can bind complementary to microRNA-206.

### BACKGROUND OF THE INVENTION

### Field of the Invention

The present invention relates to a method for analyzing the diagnosis or prognosis of a specific disorder, a diagnosis of disorder thereof and a pharmaceutical composition for targeting a specific miRNA.

### Description of the Prior Art

Among the mood disorders, depression is a mental illness that the prevalence thereof is the highest with 7 to 12% for risen and 20 to 35% for women. Depression is characterized by various symptoms such as sadness, helplessness, unhappiness, and other brain dysfunctions, with 20 to 50% recurrence within 2 years and 50 to 70% recurrence within 5 years. In addition, depression is the leading cause of Years of Life Lived with Disability (YLDs), and WHO estimates that depression will be the most important health problem in the world for decades to come.

A suicide risk in the depressed patients is elevated 10 fold or more,and 80% of suicides are associated with depression, or 33% of them are known to be due to direct depressive symptoms. The diagnosis of depression is based on phenotypic symptoms and follows the criteria of the Diagnostic and Statistical Manual of Mental Disorders (DSM-IV-TR) as presented by the AmerPsychiatric Association or the International Classification of Diseases (ICD-10) of the WHO. Up to date, there is no biological diagnostic tool for depression that has been put to practical use.

Despite of the development of various therapies over the last decades, more than 50% of patients with depression have had poor therapeutic response, and more than 35% have shown therapeutic resistance. In animal studies using genetic mutations, while a correlation between antidepressant treatment and changes of nerve related biomarkers such as brain-derived amyotrophic factor (BNF) is continuously reported, genetic studies fail to yield significance of most candidate genes. Therefore, it is necessary to develop therapeutic and develop diagnostic tools or more radical treatment rather than genetic approaching method.

### SUMMARY OF THE INVENTION

Accordingly, it is an object of the present invention to provide a method of detecting a specific miRNA for analyzing the diagnosis or prognosis of a mental illness by a mood disorder, a method for providing information for the diagnosis of a mental illness by a mood disorder, and a diagnostic composition and a pharmaceutical composition for a mental illness by a mood disorder.

According to an embodiment of the present invention in order to achieve the above object, it is characterized in that the present invention relates to a method for detecting diagnostic or prognostic marks of a mental illness by a mood disorder by using a method of detecting mire-206 expression to provide information necessary for diagnosis or prognosis analysis of a mental illness by a mood disorder.

Furthermore, it is characterized in that the present invention relates to a method of providing information for the diagnosis of a mental illness by a mood disorder comprising the step of measuring the expression level of mire-206 from a tissue sample of a patient and comparing the expression level of said mire-206 with a reference level.

In addition, it is characterized in that the present invention relates to an agent for measuring the expression level of mire-206, a composition for diagnosing a mental illness by a mood disorder, which comprises a primer pair, a probe or an antisense nucleotide capable of complementarily binding to the said miR-206.

And, it is characterized in that the present invention relates to a pharmaceutical composition for treating or preventing a mental illness by a mood disorder, which is capable of inhibiting the activity of miR-206.

Being constituted as the above, the diagnostic method of the mental illness by a mood disorder according to an embodiment of the present invention can be diagnosed more accurately by comparing the relative expression level of miR-206, and furthermore, can be analyzed prognosis of the mental illness by a mood disorder by using a substance targeting miR-206 as a marker.

Further, the method for providing information for diagnosing the mental illness by a mood disorder according to the present invention can be more efficiently analyzed.

In addition, the composition for diagnosing a mental illness by a mood disorder which targets miR-206 according to the present invention can reduce the misdiagnosis rate for a mental illness by a mood disorder, and it is a pharmaceutical composition targeting miR-206 which enables to enhance the effectiveness of the treatment and to minimize side effects of the treatment by providing a pharmaceutical composition for essential treatment or prophylaxis for a mental illness by a mood disorder.

The effects of the present invention described above are merely one of various effects according to the present invention, and the present invention can be realized in various forms according to the application mode of the embodiment.

### BRIEF DESCRIPTION OF THE DRAWINGS

The present invention will now be described by way of example only, with reference to the accompanying drawings, in which:

FIG. 1 is a bar graph comparing the expression of IRNA-206 with the quantitative real-time polymerase chain reaction (art-PC) in the brain of depression model and normal mouse.

FIG. 2 shows a bar graph of the results of behavioral experiments measuring anxiety through an elevated plus maze experiment.

FIG. 3 shows a bar graph of the activity of the rats entering each arm in an elevated plus maze experiment.

FIG. 4 shows a bar graph of the result of performing the tail suspension test (TS) measuring the despair.

FIG. 5 shows the results of a TST experiment in a depressive model induced by chronic foot-shock stress (CF).

### DETAILED DESCRIPTION OF THE INVENTION

Hereinafter, the present invention will be described in further detail with reference to preferred embodiments. It is to be understood, however, that the scope of the present invention is not limited to these embodiments. It should be noted that the embodiments of the present invention described below are intended to sufficiently convey the spirit of the present invention to those skilled in the art.

The sequence of miR-206 according to the present invention is a human-derived sequence, and the maturation sequence thereof is 5′-UGF GAGA G-3′. The pharmaceutical composition for the treatment and prevention of the mental illness by a mood disorder according to the present invention can bind to the said miR-206 as a target, and there may be a complementary sequence with from 1^{st} to 8^{th}, from 2^{nd} to 8^{th}, or from 2^{nd} to 7^{th} nucleotide sequence among the mature sequence of the antisense miR-206 used as the effective ingredient of the said pharmaceutical composition. The most preferred sequence of the antisense oligonucleotide used as an effective ingredient in the present invention has a sequence complementary to the entire nucleotide sequence of SEQ ID NO: 1 (i.e., the mature sequence of miR-206), but is not limited thereto. The mature sequence of said miR-206 can be obtained from the miRNA database (http://www.mirbase.org).

Generally, micro RNA is transcribed into precursors of about 70-80 nt (nucleotide) long having a hairpin structure called pre-miRNA, and then produced with a matured form by cleaving with the RNAse III enzyme, Dicer. miRNAs form a ribonucleotide complex called as miRNP, which binds complementarily to the target site to cleave the target gene or inhibit translation. Over 30% of human miRNA are present with clusters, but they are transcribed into a single precursor and then cleaved to finally form matured miRNA.

The term “miRNA” or “micro RNA”, as used herein, refers to from 21 to 23 non-coding RNA that regulate gene expression after transcription by promoting degradation of target RNA or inhibiting translation thereof.

The term “substance capable of inhibiting the activity of a miRNA” as used herein includes any substance capable of inhibiting expression and/or activity. The said any substance may include, for example, an antagomir, an antisense molecule, a small hairpin RNA molecule (shRNA), a small interfering RNA molecule (siRNA), a seed target LNA (Locked Nucleic Acid) oligonucleotide, a decoy oligonucleotide, an aptamer, a ribozyme, or antibodies recognizing DNA:RNA hybrids, and the like.

In one embodiment according to the present invention, “substance capable of inhibiting the activity of a miRNA” refers to an antisense oligonucleotide which is capable of complementarily binding to all or a portion of the progenitor and/or matured sequences of miRNAs according to the present invention, particularly the seed sequence, thereby inhibiting their activity. The said inhibition of activity is to inhibit the transcription of miRNA according to the present invention and/or their binding to the target mRNA.

The said antisense oligonucleotides may or may not include one or more modifications in nucleotides constituting the antisense oligonucleotides or the backbone connecting the nucleotides. Such modifications may be included a nucleotide in which at least one nucleotide constituting the antisense oligonucleotide has LNA, or at least one 2′-O-methylated sugar, or one or more phosphothioates in the backbone.

The term “antisense oligonucleotide”, as used herein, encompasses a nucleic acid-based molecule which has a sequence complementary to all or part of the target miRNA, in particular seed sequence thereby capable of forming a duplex with a miRNA. Thus, the term “antisense oligonucleotide”, as used herein, may be referred to as “complementary nucleic acid-based inhibitor”.

The said antisense oligonucleotides can include a variety of molecules such as ribonucleic acid (RNA), deoxyribonucleic acid (DNA), antagomir, 2′-O-modified oligonucleotides, phosphorothioate-backbone deoxyribonucleotides, phosphorothioate-backbone ribonucleotides, peptide nucleic acid (PNA) oligonucleotide or LNA (Locked Nucleic Acid) oligonucleotide, and the like.

Preferably, the ribonucleic acid (RNA) may be used, and the ribonucleic acid (RNA) may include double-stranded siRNA (small interfering RNA), shRNA (short hairpin RNA), and ribozyme.

The said LNA (Locked Nucleic Acid) is a modified ribonucleotide that comprises an additional bridge between the 2′ carbon and the 4′ carbon at the ribose sugar region and has a locked form, whereby the oligonucleotide having LNA has improved thermal stability.

The said PNA (Peptide Nucleic Acids) comprise a peptide-based backbone in place of the sugar-phosphate backbone in the polynucleotide. The 2′-O-modified oligonucleotides are preferably 2′-O-alkyl oligonucleotides, more preferably 2′-O-C₁₋₃ alkyl oligonucleotides, and most preferably 2′-O-methyl oligonucleotides.

The said antisense oligonucleotides include antisense oligonucleotides, antagomir and inhibitory RNA molecules in the narrow sense.

The term “antagomir”, as used herein, means the chemically modified oligonucleotides of a single strand, and are used for silencing of endogenous miRNA. The said antagomir may contain a sequence that is not complementary at the Arganoute 2 (Ago 2) cleavage site, or inhibit cleavage of Ago 2 such that the base is modified with, for example, 2′ methoxy group, 3′ cholesterol group, or a phosphorothioate. There is a complementary sequence to the target sequence.

The antagomir according to the present invention may have a sequence which is at least partially or completely complementary to an miRNA according to the invention, and in one embodiment, the antagomir includes one or more variants (for example, 2′-O-methyl-sugar modification or 3′ cholesterol modification). In other embodiments, the antagomir may comprise one or more phosphorothioate linkages and may also have a phosphorothioate backbone in part.

Suitable antagomir lengths for inhibiting the activity of miRNA according to the present invention are, but are not limited to, from 7 to 50 nt, especially from 10 to 40 nt, more particularly from 15 to 30 nt, even more particularly from 15 to 25 nt, in particular from 16 to 19 nt. For example, miRNA regulatory material having a sequence capable of complementarily binding to a miR-206 seed sequence may be included.

The term “complementary”, as used herein, means that the antisense oligonucleotide is sufficiently complementary to a target of miRNA according to the present invention under predetermined hybridization or annealing conditions, preferably under physiological conditions, and encompassing some or partial, but substantially complementary, and perfectly complementary both, and preferably means completely complementary. Substantially complementary means that although not perfectly complementary, it has complementarities sufficient to bind to the target sequence so that have an effect according to the present invention, namely sufficient effect to interfere with the activity of the miRNA.

The term “nucleic acid”, as used herein, may include polynucleotides, oligonucleotides, DNA, RNA, and analogs and derivatives thereof, and may include, for example, peptide nucleic acid (PNA) or mixtures thereof In addition, the nucleic acid can be single or double stranded and can encode molecules including polypeptides, mRNA, miRNA or siRNA, and the like.

In one embodiment of the invention, the antisense oligonucleotides or nucleic acid molecules described herein comprise sequences complementary to all or part of the miRNA seed sequence according to the present invention. The seed sequences are the conserved sequences in a variety of species that are critical to the recognition of target molecules in miRNA. Since miRNA binds to the target through the seed sequence, it can effectively inhibit translation of the target mRNA and the like when the interaction between the seed sequence and the target is inhibited.

The pharmaceutical composition according to the present invention is provided for the treatment or prevention of a mental illness by a mood disorder and may be used effectively for treating or preventing the disease by administering an effective amount of the pharmaceutical composition to a subject in need of treatment or prevention of the disease ultimately.

The terms “treatment”, “alleviation” or “amelioration”, as used herein, refer to any behavior that improves or alters beneficially the symptoms of the related mental disorders by administration of the said pharmaceutical composition. Those skilled in the art will be able to know the precise criteria of the diseases by referring to the data presented by the Korean Medical Association, and can judge the degree of improvement, improvement, and treatment of the diseases.

In addition, as used herein, the term “prevention” refers to any behavior that inhibits or delays the onset of the related mental disorders. It will be apparent to those skilled in the art that compositions of the present application can prevent the related mental disorders with being administered when they exhibit initial symptoms or prior to presentation of the initial symptoms.

In addition, the pharmaceutical composition according to the present invention may further comprise at least one active ingredient exhibiting the same or similar function in the treatment of a mental illness by a mood disorder in addition to a substance capable of inhibiting the activity of the said miRNA, or may further comprise a compound that maintains/increases solubility and/or absorbency of active ingredient. In addition, it may further optionally include a chemotherapeutic agent, an anti-inflammatory agent, an antiviral agent, and/or an immunemodulator.

In addition, the said pharmaceutical composition may further comprise one or more pharmaceutically acceptable diluents, carriers and/or adjuvants in addition to the above mentioned active ingredients. The said pharmaceutically acceptable carrier may comprise one or more of saline, sterile water, Ringer's solution, buffered saline, dextrose solution, maltodextrin solution, glycerol, ethanol, liposome, a mixture thereof, and if necessary, conventional additives such as antioxidants, buffers, bacteriostatic agent and the like may be further included. In addition, it can be formulated into injectable formulations such as aqueous solutions, suspensions, emulsions and the like, pills, capsules, granules or tablets by additionally adding diluents, dispersants, surfactants, binders and lubricants, and it can be used by binding a target organ specific antibody or other ligand with the said carrier to be able to act specifically on the target organ.

Furthermore, it can be suitably formulated according to each disease or ingredient by using appropriate methods in the art or using the methods disclosed in the Remington's literature. For example, the said pharmaceutical compositions may be prepared in a variety of formulations and dosage forms, such as solutions, emulsions and liposome formulations, and may be prepared in the forms of tablets, capsules, gels, syrups or suppositories by mixing the active ingredient with a pharmaceutically acceptable liquid and/or finely divided solid carrier or excipient.

In addition, the said pharmaceutical composition may be prepared as a suspension using an aqueous, non-aqueous or mixed medium. The aqueous suspensions may further comprise substances that increase the viscosity of the suspension such as sodium carboxymethyl cellulose, sorbitol and/or dextran.

In the present invention, the method of administering the said pharmaceutical composition is not particularly limited, and a known method of administering the inhibitor may be applied. The said pharmaceutical composition may be administered parenterally (for example, intranasal, intravenous, subcutaneous, intraperitoneal or topical administration) or orally, depending on the intended method. In order to obtain a rapid therapeutic effect, intranasal administration is preferred. In addition, the said pharmaceutical composition may be delivered in various routes, for example, via infusion or bolus injection, epidermis or mucosa (oral mucosa, anal mucosa, intestinal mucosa, etc.), and may be administered systemically or topically.

It is also preferred that the said pharmaceutical composition is introduced into the central nervous or peripheral nerves through an appropriate route. Suitable routes may include intraventricular or intrathecal administration. Such administration can be accomplished using a catheter connected to the reservoir. Also, it can be formulated as an aerosol and administered via the lungs via an inhaler or sprayer, but does not exclude intravenous administration, subcutaneous injection, intracerebroventricular injection, inhalation administration, or oral administration.

In one embodiment of the invention, the pharmaceutical composition is preferably administered intranasally. When the said pharmaceutical composition is administered into the nasal cavity, it is delivered to the brain along the olfactory nerve pathway, thereby enhancing the effect of the pharmaceutical composition. Acceptable carrier for intranasal administration of the said pharmaceutical composition may be comprised, which the carrier comprises one or more suitable solid, filler diluent or encapsulating material suitable for administration to any part of nasal epithelium of the mammalian, preferably human. Typically, the said carrier may be a liquid, solution, suspension, gel, ointment, lotion, or a combination thereof, preferably an aqueous carrier.

In addition, the said pharmaceutical composition may be prepared in various unit dosage forms. Such forms include, but not be limited to, nasal drops, nasal sprays, nasal gels, nasal ointments, and nasal powders.

The said carrier may comprise a delivery enhancer wherein the intranasal delivery enhancer may include an aggregation inhibitor, a dosage modifier, a pH controlling agent, a degradative enzyme inhibitor, a mucolytic dissolution or mucolytic agent, ciliostatic reagents, a membrane permeation enhancer (for example, a surfactant, a bile salt, a phospholipid or a fatty acid additive, a mixed micelle, a liposome, or a carrier, an alcohol, an enamine, a nitric oxide donor mixture, a long-chain amphipathic molecule, small hydrophobic penetration enhancer, sodium or salicylic acid derivatives, glycerol esters of acetoacetic acid, cyclodextrin or beta-cyclodextrin derivatives, mesochain fatty acids, chelating reagents, amino acids or salts thereof, N-acetylamino acids or salts thereof, degrading enzymes for selected membrane components, fatty acid synthesis inhibitor, a cholesterol synthesis inhibitor or a nitric oxide stimulating substance, a chitosan and modulating agents of epithelial junctional physiology such as chitosan derivatives, a vasodilator, a selective transport promoter and, stable delivers, carriers, and support materials that the said pharmaceutical composition can be effectively combined, bound, stored or encapsulated, or allow to stabilize the active agent to enhance intranasal mucosal delivery, or complex-forming species.

As used herein, the term “pharmaceutically or therapeutically effective amount” means an amount sufficient to treat a disease at a reasonable benefit/risk ratio applicable to medical treatment, and the effective dose level will depend on the type of disease, severity, activity of the drug, sensitivity to the drug, the time of administration, the route of administration and the rate of release, the duration of the treatment, factors including drugs used at the same time, and other factors well known in the medical arts.

The said pharmaceutical composition may be administered as an individual therapeutic agent or in combination with other therapeutic agents, sequentially or concurrently with conventional therapeutic agents, and may be administered singly or multiply. It is most important that the said pharmaceutical composition is administered in such an amount that the maximum effect can be obtained in a minimum amount without side effects considering all of the above mentioned factors, which can be easily determined by those skilled in the art.

Also, the dosage range of the said pharmaceutical composition may vary depending on the patient's body weight, age, sex, health condition, diet, administration time, administration method, excretion rate, severity of disease, and the like, and the proper dosage amount may also be varied depending on the amount of the drug accumulated in the patient's body and/or the specific effectiveness degree of the polynucleotide used. In general, it can be calculated on the basis of the the EC₅₀ (half maximal effective concentration) measured as effective at in vivo animal model and in-vitro conditions. For example, it may be from 0.01 μg to 1 g per 1 kg of body weight, and may be administered once to several times per unit period in a daily, weekly, monthly, or annual unit period. In addition, it can be continuously administered for a long period of time with the infusion pump, and the number of times of repeated administration can be determined in consideration of the time during which the drug remains in the body, the drug concentration in the body, and the like. Even after treatment according to the course of the disease treatment, the said pharmaceutical composition can be continuously administered to prevent the recurrence of the disease.

In the present invention, an antisense oligonucleotide may be contained as an active ingredient related to a mental illness by a mood disorder that may be further included in the said pharmaceutical composition, and the said antisense oligonucleotide may be further included as itself or in a form of a pharmaceutically acceptable salt in the said pharmaceutical composition.

The said pharmaceutically acceptable salt means that the biological activity of the polynucleotide is maintained while the toxicity is minimized. Such pharmaceutically acceptable salts include, but are not limited to, a base addition salt formed with metal cation such as zinc, calcium, bismuth, barium, magnesium, aluminum, copper, cobalt, nickel, cadmium, sodium, potassium, and the like, salts formed with organic amino acids, salts formed with cations derived from ammonia, N,N-dibenzylethylene-diamine, D-glucosamine, tetraethylammonium or ethylenediamine.

Meanwhile, the method of providing information for the diagnosis of the mental illness by a mood disorder according to the present invention is a process of confirming the presence and expression strength of miR-206 in a sample, and by measuring the amount of miR-206, whether the level of expression is increased or decreased can be ascertained. In the present invention, measurement of expression levels involves measurement of miRNA-206 expression levels. Examples of the assay method include, but are not limited to, reverse transcriptase polymerase, competitive reverse transcriptase polymerase, real-time reverse transcriptase polymerase, RNase protection assay, Northern Blotting or DNA chip.

The above mentioned diagnostic method may further include the clinical information of the subject together with the presence or absence of an increase in the expression level of miR-206 to determine the onset of the mental illness by a mood disorder. Such clinical information of the subject includes, but is not limited to, at least one of the age, sex, body weight, dietary habit, body mass index, underlying disease, blood test result, brain MRI result, brain CT result, or cerebrospinal fluid test result of the test subject.

The mental illness by a mood disorder according to the present invention include, but are not limited to, depression, dysthymic disorder, depressive disorder, short-term depressive disorder, depressive disorder before menstrual and bipolar disorder.

And, in the present invention, the diagnosis include determining susceptibility of the subject to a specific disease or a disease, determining whether the subject currently has a particular disease or disorder, determining the prognosis of the particular disease or disordered subject, or therametrics (e. g., monitoring the status of the subject to provide information about the therapeutic efficacy).

The experimental steps used for embodiments of the present invention can be described as follows.

1. Preparation of Animal Models with Depression

C57BU6, a member of the B6 mice, was used with animal models and matched both the age and gender of the normal and experimental groups. The depression model was produced by the following two methods.

1) Depression Model by Corticosterone (CORT) Injection

35 μg/mL of CORT (equivalent to 5 mg/kg/day, Sigma; St. Louis, Mo., USA) was treated with water containing 0.45% β-cyclodextrin (β-CD; Sigma). And it was fed for 42 days to normal mice, and CORT treated water was replaced every 3 days. In addition, water bottles that light was blocked were used.

2) Chronic Foot-Shock Stress (CFS) Depression Model

Normal mice were given foot-shocks of 0.3-mA for 2 seconds during 1 hour respectively for 4 weeks. Foot-shocks were randomly assigned at an interval of 2 to 15 seconds so as to be unpredictable.

2. Real-Time Quantitative PCR (qRT-PCR) of miRNA

The expression level of miR-206 was examined using qRT-PCR (reverse transcriptase quantitative PCR). The miRNA were isolated from the brains of control group (normal mouse) and experimental group (CORT induced depression model and CFS induced depression model) using Ambion mirVana isolation kit. Real-time PCR was performed using probes and primers included in TaqMan MicroRNA Assays (Life Technologies, USA) according to the manufacturer's method. It was then analyzed using ABI PRISM 7000 sequence detection system (Ambion-Applied Biosystems, USA). The conditions were performed repeatedly 40 cycles with heat treatment at 50° C. for 2 minutes and 95° C. for 10 minutes, followed by heat treatment at 95° C. for 15 seconds and 60° C. for 60 seconds, and all reactions were done in triplicate. The relative expression degree was calculated using the comparative threshold cycle (Ct) 2^{-ΔCt} equation and the concentration of miRNA was determined by normalization to the snoRNA-202 concentration measured using an endogenous snoRNA detection kit (Ambion-Applied Biosystems).

Preferably one or more oligonucleotides capable of complementarily binding with miR-206 for the quantitative detection of miR-206 were included, wherein a primer, a reverse transcriptase, a Taq polymerase, a primer for PCR and dNTP corresponding to a part of the sequence may be also included.

3. Inhibition of miR-206

Inhibition of miRNA-206 was conducted with using a antagomir (AM-206, 2′-O-methylated antisense oligonucleotide, sequence: 5′-cca cac acu ucc uua cau ucc a-3′, Bionear Co., Korea) sequence-specific for miRNA-206. After the mouse was anesthetized, AM206 (5 nmol of distilled water treated with of 0.1% v/v diethylpyrocarbonate) was administered into the nasal cavity by pipetting in 4 of each nostril every 2 minutes (total 6 fractions). Control mice were given equal volume of distilled water (vehicle) treated with diethylpyrocarbonate. AM-206 was administered once a day for 3 days.

4. Behavioral Experiments

One week after administration of AM-206 and vehicle, behavioral experiments were performed on the depressed animal models. Behavioral experiments were performed with EPM (Elevated plus maze) experiment for anxiety measurement and TST (Tail suspension test) experiment for measuring hopelessness, and all behavioral experiments were recorded on video.

1) Elevated Plus Maze (EPM)

EPM is made of acrylic and consists of a cross(+) maze at 40 cm above the floor. Two of the four pathways facing the maze are open (open aim) and two opposite pathways are closed by a 20 cm high wall (closed aim). The center platform is 8 cm of horizontal and 8 cm vertical wide, allowing the maze to be freely navigated for five minutes with the mouse on the central platform at the start of the experiment. And then the time spent on the open and closed arms of the mouse and the number of times of each aim was analyzed.

2) Tail Suspension Test (TST)

About 1 cm from the tail tip of the mouse was taped to the middle of the horizontal bar at a height of 50 cm, and the immobility duration was measured for 6 minutes.

5. Statistical Analysis

A comparison of the results of the control group (normal mouse) and experimental group (CORT depression induced model group and CFS depression induced model group) was done by Student's T test and a comparison of the results of the control group (normal mouse), experimental group 1 (CORT depression induced model group) and experimental group 2 (CFS depression induced model group) were performed by using the one-way ANOVA method. All results were expressed as mean±standard errors. For statistical analysis, SPSS 21.0 (SPSS Inc, Chicago, Ill.) was used, and the P-value was significantly considered when being less than 0.05.

### EXAMPLE 1

### Upward Expression of miR-206 in Brain Tissue of Depression Induced Mouse Model

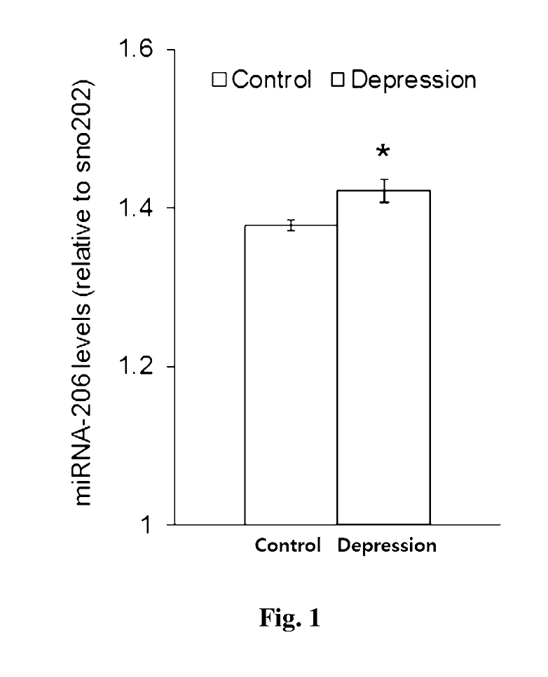

The above experiment was carried out in substantially the same manner and shown the result quantitatively analyzed for an amount of expression of miRNA-206 in the brain tissues of the experimental group (CORT depression induction model (n=3) and CFS depression induced mouse model (n=5)) and control group (normal mouse) (n=5) by qRT-PCR (quantitative real-time polymerase chain reaction) in FIG. 1 as a bar graph. The abscissa of the bar graph in FIG. 1 represents the control group and the experimental group, and the ordinate represents the normalized value for an amount of expression of miR-206 in snoRNA-202.

Referring to the bar graph of FIG. 1, it was confirmed that miRNA-206 was upward expressed in the mice of the experimental group (CORT induced depression model and CFS induced depression model) more than the control group (normal mouse). The snoRNA-202 was used as an internal control and the value of bar graphs were normalized to the expression of miRNA-206 in snoRNA-202 (control, 1.38±0.01; depression, 1.42±0.01, *P<0.01, the bar means±standard error).

### EXAMPLE 2

### Convalescence from Anxiety in CORT Induced Depression Model Due to Administration of AM-206

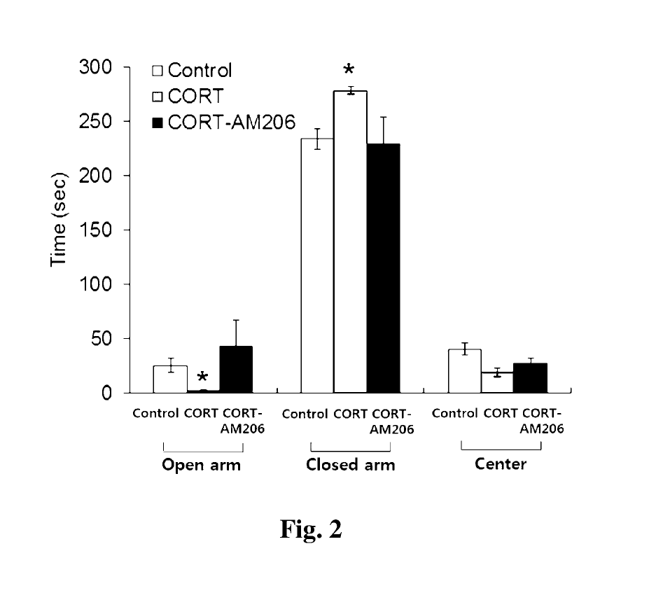

The above experiment was carried out in substantially the same manner to measure the depression level of the control group and the experimental group 1 (CORT induced depression model group) via the EPM experiment, and the results are shown in a bar graph in FIG. 2. The abscissa of the bar graph in FIG. 2 represents the open aim, the closed arm and the center platform, and the ordinate refers to the time (sec) in each passage.

Referring to the bar graph of FIG. 2, it can be shown that experimental group 1 (CORT induced depression model group) spent more time in the closed aim than the control group (control, 233.93±9.12; CORT, 278.76±3.85 ; CORT-AM206, 229.20±25.16 sec), and less time in the open aim (control, 25.65±6.25; CORT, 1.98±0.96; CORT-AM206, 43.61±23.56 sec). From these results, it can be confirmed that mice of the CORT induced depression model show high anxiety.

However, it can be confirmed that mice of the CORT induced depression model (CORT-AM206) in which the antisense oligonucleotide AM-206 (antagomir for miR-206) against miRNA-206 was administered spent a similar time compared to the control group (normal mice).

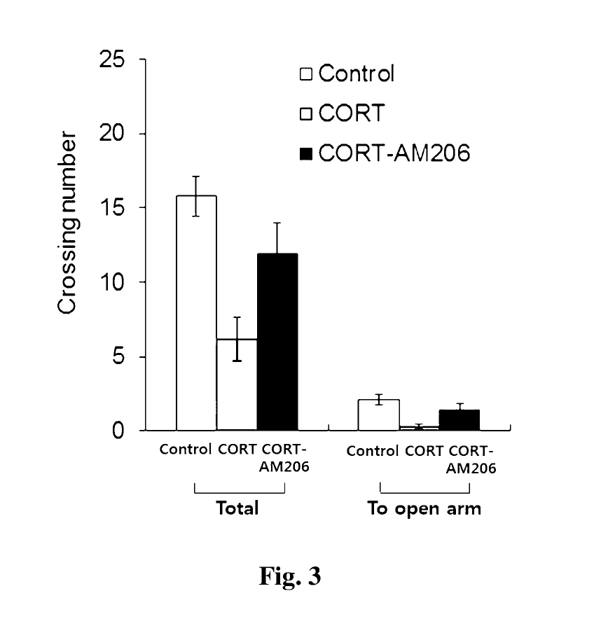

Meanwhile, FIG. 3 is a bar graph showing the number of times entering each of the arms (activity). The abscissa of the bar graph of FIG. 3 refers to all (total) passage directions and to open aim passage directions, and the ordinate refers to the number of times entering into the passage.

Referring to the bar graph of FIG. 3, it can be confirmed that the reduced total number of times in the experimental group 1 (CORT induced depression model) compared to control group (control, 15.80±1.34; CORT, 6.18±1.49; CORT-AM206, 11.90±2.09) and the number of times entering into the open arm (activity) were recovered in the CORT-AM206 group (control, 2.10±0.35; CORT, 0.29±0.14; CORT-AM206, 1.4±0.43) (control group n=10, CORT induced depression model group n=17, CORT-AM206 group n=10, *P<0.05, one-way ANOVA, the bar means±standard error).

Thus, in FIGS. 2 and 3, it can be confirmed that AM-206 restores high anxiety in the depression model.

### EXAMPLE 3

### Recovery of Despair of CORT Depression Model Due to AM206

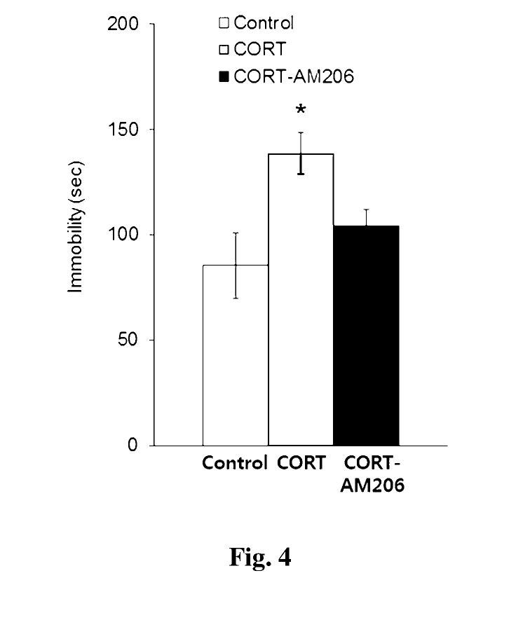

The above experiment was carried out in substantially the same manner to measure the degree of despair of the control group (normal mouse) and the experimental group 1 (CORT induced depression model group) via the TST experiment, and the results are shown in a bar graph in FIG. 4. The abscissa of the bar graph in FIG. 4 represents the control group, the experimental group 1 (CORT induced depression model), CORT-AM206 group, and the ordinate refers to time (sec) in which immobility occurs.

It can be confirmed that via the bar graph of FIG. 4, the experimental group 1 (CORT induced depression model) is more desperate than the control group through immobility for a longer time than the control group. However, it can be confirmed that the CORT-AM206 group showed similar values to the control group (normal mouse) (control, 85.30±15.46; CORT, 138.60±9.74, CORT-AM206, 104.33±7.53 sec). From these results, it can be confirmed that AM-206 restores the high despair of the CORT-induced depression model (control n=14, CORT-induced depression model group n=12, CORT-AM206 group n=9, 0.05, one-way ANOVA, the bar means±standard error).

### EXAMPLE 4

### Recovery of Despair of CFS Induced Depression Model Due to AM206

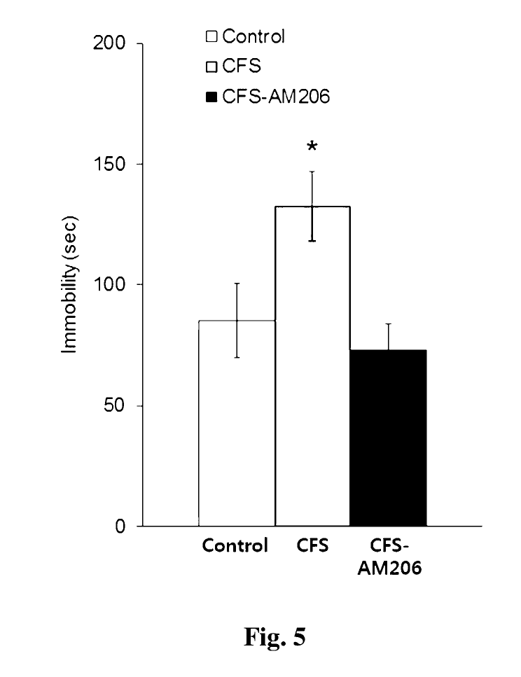

The above experiment method was practically carried out to measure the degree of despair in the experimental group 2 (CFS induced depression model) through the TST test, and the results are shown in a bar graph in FIG. 5. The abscissa of the bar graph in FIG. 5 represents the control group, the experimental group 2 (CFS induced depression model), and the CFS-AM206 group, and the ordinate refers to time (sec) in which immobility occurs.

It can be confirmed that via the bar graph of FIG. 5, the experimental group 2 (CFS induced depression model) is more desperate than the control group through immobility for a longer time than the control group, but the CFS-AM206 group administered AM-206 showed similar values to the control group (control, 85.30±15.46; CFS, 132.14±14.42; CFS-AM206, 73.61±10.55 sec). From this result, it can be confirmed that AM-206 restores the high despair of the CFS induced depression model (control n=14, CFS induced depression model n=20, CFS-AM206 group n=12, * P <0.05, one-way ANOVA, the bar is±standard error).

Although the present invention has been particularly described with reference to exemplary embodiments thereof as above, it is to be understood that the invention is not limited to the disclosed embodiments, but is intended to cover various modifications and equivalent arrangements carried out by the person skilled in the art and included within the spirit and scope of the appended claims.

Unless otherwise defined, all technical terms used in the present invention are used in the sense that they are generally understood by the person skilled in the relevant field of the present invention.

What is claimed is:

1. A method for detecting diagnostic or prognostic marks of a mental illness by a mood disorder by using a method of detecting milt-206 expression to provide information necessary for diagnosis or prognosis analysis of a mental illness by a mood disorder.

2. The method for detecting diagnostic or prognostic marks of a mental illness by a mood disorder according to claim 1, wherein the said detecting miR-206 expression utilize a primer pair, a probe or an antisense nucleotide capable of complementarily binding to the said miR-206.

3. The method for detecting diapostic or prognostic marks of a mental illness by a mood disorder according to claim 1, wherein the said method of detecting milt-206 expression is the method using any one of reverse transcriptase polymerase, competitive reverse transcriptase polymerase, real-time reverse transcriptase polymerase, RNase protection assay, Northern Blotting or DNA chip.

4. The method for detecting diapostic or prognostic marks of a mental illness by a mood disorder according to claim 1, wherein the said mental illness by a mood disorder is depression, dysthymic disorder, depressive disorder, short-term depressive disorder, depressive disorder before menstrual and bipolar disorder.

5. A method of providing information for the diagnosis of a mental illness by a mood disorder comprising the step of:
measuring the expression level of miR-206 from a tissue sample of a patient; and
comparing the expression level of the said miR-206 with a reference level.

6. The method of providing information for the diagnosis of a mental illness by a mood disorder according to claim 5 further comprising the step of comparing the expression level of the said miR-206 with a reference level, and determining with a mental illness by a mood disorder when the said expression level is increased.

7. The method of providing information for the diagnosis of a mental illness by a mood disorder according to claim 5, wherein the said comparing step further uses at least one clinical information of age, sex, weight, diet, body mass, underlying disease, blood test result, brain MRI result, brain CT result or brain spinal fluid test result of the patient to determine a mental illness by a mood disorder.

8. The method of providing information for the diagnosis of a mental illness by a mood disorder according to claim 5, wherein the said expression level of miR-206 is measured by utilizing a primer pair, a probe or an antisense nucleotide capable of complementarily binding to the said miR-206.

9. The method of providing information for the diagnosis of a mental illness by a mood disorder according to claim 5, wherein the said method of measuring expression level of miR-206 is the method using any one of reverse transcriptase polymerase, competitive reverse transcriptase polymerase, real-time reverse transcriptase polymerase, RNase protection assay, Northern Blotting or DNA chip.

10. An agent for measuring the expression level of miR-206 which is a composition for diagnosing a mental illness by a mood disorder comprising a primer pair, a probe or an antisense nucleotide capable of complementarily binding to the said miR-206.

11. The kit for diagnosing a mental illness by a mood disorder comprising the composition for diagnosing according to claim 10.

12. A pharmaceutical composition for treating or preventing a mental illness by a mood disorder targeting miR-206.

13. The pharmaceutical composition for treating or preventing a mental illness by a mood disorder according to claim 12, wherein the substance targeting the said miR-206 comprises a nucleic acid molecule capable of binding to all or part of the nucleotide sequence of SEQ ID NO: 1 of the said miR-206.

14. The pharmaceutical composition for treating or preventing a mental illness by a mood disorder according to claim 13, wherein the said nucleic acid molecule is RNA, DNA, antagomir, antisense molecules, siRNA, shRNA, 2′-O-modified oligonucleotide, phosphorothioate-backbone deoxyribonucleotide, phosphorothioate-backbone ribonucleotide, decoy oligonucleotide, PAN (peptide nucleic acid) oligonucleotide or LNA (locked nucleic acid) oligonucleotide.

15. The pharmaceutical composition for treating or preventing a mental illness by a mood disorder according to claim 12, wherein the composition targeting the said miR-206 is used for producing an agent capable of treating or preventing a mental illness by a mood disorder through an anxiety-restoring action.

16. The pharmaceutical composition for treating or preventing a mental illness by a mood disorder according to claim 12, wherein the composition targeting the said miR-206 is used for treating or preventing a mental illness by a mood disorder through an desperation-restoring action.

17. The pharmaceutical composition for treating or preventing a mental illness by a mood disorder according to claim 12, wherein the composition targeting the said miR-206 is utilized for producing an agent for treating or preventing a mental illness by a mood disorder through an action of inhibiting the expression or activity of miR-206.


SEQUENCE LISTING (1 sequence)

SEQ ID NO: 1 - RNA, 22 nt, Homo sapiens
uggaauguaaggaagugugugg